(19) United States
(12) Patent Application Publication
(10) Pub. No.: US 2014/0056948 A1
(43) Pub. Date: Feb. 27, 2014

(54) HARD TISSUE REGENERATION MATERIAL AND METHOD FOR MANUFACTURING THE SAME

(71) Applicant: National Cheng Kung University, (US)

(72) Inventors: Dar-Bin SHIEH, Tainan City (TW); Hsin-Ju CHANG, Kaohsiung City (TW); Hung-Ching LU, Tainan City (TW)

(73) Assignee: NATIONAL CHENG KUNG UNIVERSITY, Tainan City (TW)

(21) Appl. No.: 13/772,429

(22) Filed: Feb. 21, 2013

Related U.S. Application Data
(60) Provisional application No. 61/602,129, filed on Feb. 23, 2012.

(30) Foreign Application Priority Data
Feb. 23, 2012 (TW) 101137418

(51) Int. Cl. A61K 6/00; A61L 27/02

(52) U.S. Cl. CPC A61K 6/0073; A61L 27/025
USPC 424/400; 424/641

(57) ABSTRACT

A hard tissue regeneration material and a method for manufacturing the same are disclosed. The hard tissue regeneration material of the present invention comprises: ZnO particles selected from the group consisting of crystallized ZnO particles, crystallized ZnO nanorods, nano-ZnO hollow fibers, and a combination thereof; and at least one selected from the group consisting of polycarboxylate cement, glass ionomer cements, and collagen.

### CROSS REFERENCE TO RELATED APPLICATION

This application claims the benefits of the Taiwan Patent Application Serial Number 101137418, filed on Oct. 11, 2012, the subject matter of which is incorporated herein by reference.

This application claims the benefit of filing date of U.S. Provisional Application Ser. No. 61/602,129, entitled “ZnO, Teeth Cleaning Composition, and Hard Tissue Regeneration Material Containing the Same” filed Feb. 23, 2012 under 35 USC §119(e)(1).

### BACKGROUND OF THE INVENTION

1. Field of the Invention

The present invention relates to a hard tissue regeneration material and a method for manufacturing the same and, more particularly, to a regeneration material for tooth or bone repair and a method for manufacturing the same.

2. Description of Related Art

There are a lot of conventional hard tissue regeneration material, in which a desirable hard tissue regeneration material should meet the requirements of easily-shaping, non-toxicity, chemical stability, good biocompatibility, and enough mechanical strength.

One conventional hard tissue regeneration material is glass ionomer, which can release fluoride ions and bond with dental matrix, and have good biocompatibility to pulp tissue; so the glass ionomer is one common regeneration material used in clinic. Especially, for filling class V dental caries, glass ionomer has good biocompatibility and adhesion to periodontal tissue, so it can also be used for pulp or periodontal tissue repair. However, some studies indicated that conventional glass ionomer materials show good biocompatibility to periodontal fibroblastic cells and epithelial cells, but poor biocompatibility to other dental materials such as resin-modified materials. In addition, when the glass ionomers release chemical materials or the pH of the surroundings is changed, the toxicity of the glass ionomer may be increased. Hence, some doubts about the biocompatibility and toxicity of the glass ionomers still exist.

On the other hand, for the dental repair, not only the biocompatibility and the toxicity but also the anti-bacterial property of the repairing materials has to be considered, in order to prevent inflammation in periodontal tissues caused by bacteria infection. In addition, the curing time and the mechanical strength of the regeneration material is also one factor to determine whether the material is a suitable dental material or not.

Hence, it is desirable to develop a hard tissue regeneration material, which can be applied to both the bone repair and dental repair.

### SUMMARY OF THE INVENTION

An object of the present invention is to provide a method for manufacturing a hard tissue regeneration material which has excellent biocompatibility and mechanical strength.

Another object of the present invention is to provide a hard tissue regeneration material, which has not only excellent biocompatibility but also good anti-bacterial effect. Hence, the hard tissue regeneration material of the present invention is suitable for tooth or bone regeneration.

To achieve the aforementioned objects, the method for manufacturing the hard tissue regeneration material, which comprises the following steps: (A) providing a curing material and ZnO particles wherein the curing material is at least one selected from the group consisting of polycarboxylate cement, glass ionomer cement, and collagen, the ZnO particles are at least one selected from the group consisting of crystallized ZnO nanoparticles, crystallized ZnO nanorods, and nano-ZnO hollow fibers, diameters of the crystallized ZnO nanoparticles are 25 nm-200 nm, cross-sectional diameters of the crystallized ZnO nanorods are 50 nm-1000 nm, the nano-ZnO hollow fibers have tube-like structure, and cross-sectional diameters of the nano-ZnO hollow fibers is 500 nm-3 μm; and (B) mixing the curing material and the ZnO particles to form a hard tissue regeneration material.

After the aforementioned process, the hard tissue regeneration material of the present invention can be obtained, which comprises: a curing material, which is at least one selected from the group consisting of polycarboxylate cement, glass ionomer cement, and collagen; and ZnO particles. In the hard tissue regeneration material of the present invention, the ZnO particles can be at least one selected from the group consisting of crystallized ZnO nanoparticles, crystallized ZnO nanorods, and nano-ZnO hollow fibers, wherein diameters of the crystallized ZnO nanoparticles are 25 nm-200 nm, cross-sectional diameters of the crystallized ZnO nanorods are 50 nm-1000 nm, the nano-ZnO hollow fibers have tube-like structure, and cross-sectional diameters of the nano-ZnO hollow fibers is 500 nm-3 μm. Herein, each the nano-ZnO hollow fiber is preferably composed of plural ZnO nanoparticles with diameters of 20 nm-200 nm.

In the hard tissue regeneration material and the method for manufacturing the same of the present invention, when the collagen with excellent biocompatibility and strength is mixed with nano-sized ZnO particles, the biocompatibility and the mechanical strength of the obtained hard tissue regeneration material can be improved. In addition, the obtained hard tissue regeneration material further has anti-bacterial property, so the risk of tissue infection can be decreased when the hard tissue regeneration material is used. In addition, the collagen has good adhesion to cells and can facilitate the cell growth, so the adhesion strength between the obtained hard tissue regeneration material and peripheral tissues or matrix can be improved. Furthermore, since the hard tissue regeneration material further comprises ZnO particles with anti-bacteria property, so it is suitable to be used as a tooth regeneration material. Especially, the hard tissue regeneration material of the present invention is suitable used in dental fields such as filling of class V dental caries, adhesion of crowns and/or bridges, and root canal treatment.

In the hard tissue regeneration material and the method for manufacturing the same of the present invention, each nano-ZnO hollow fibers can be composed of plural ZnO nanoparticles. Preferably, each nano-ZnO hollow fiber is composed of plural crystallized ZnO nanoparticles. In addition, the diameters of the ZnO nanoparticles can be 25 nm-200 nm.

In the hard tissue regeneration material and the method for manufacturing the same of the present invention, the crystallized ZnO nanoparticles can be single-crystallized ZnO nanoparticles, poly-crystallized ZnO nanoparticles, or a combination thereof. Preferably, the crystallized ZnO nanoparticles are single-crystallized ZnO nanoparticles.

Type I collagen is one common collagen found in extracellular matrix, and has excellent adhesion property to cells. In addition, type I collagen is a praline-rich and basic material, so it can uniformly disperse in glass ionomer cement. Hence, the collagen used in the present invention is preferably type I collagen. When the type I collagen is used in the present invention, not only the compatibility between the hard tissue regeneration material and tissues but also the mechanical strength of the hard tissue regeneration material can be improved.

In the method for manufacturing the hard tissue regeneration material of the present invention, the content of the collagen can be 0.005-2 wt % of the glass ionomer cement solution. Preferably, the content of the collagen is 0.01-1 wt % of the glass ionomer cement solution.

Hence, in the obtained hard tissue regeneration material of the present invention, the content of the collagen can be 0.005-2 wt % of the glass ionomer cement. Preferably, the content of the collagen is 0.01-1 wt % of the glass ionomer cement.

In the method for manufacturing the hard tissue regeneration material of the present invention, the polycarboxylate cement can be any polycarboxylate cement generally used for filling dental caries in the art. Preferably, the polycarboxylate cement is HY-Bond polycarboxylate cement.

In addition, in the method for manufacturing the hard tissue regeneration material of the present invention, the addition amount (or the content by weight) of the ZnO particles is not particularly limited. Preferably, the content of the ZnO particles is 0.01-40 wt % based on the total weight of the hard tissue regeneration material comprising the ZnO particles and the polycarboxylate cement. More preferably, the content of the ZnO particles is 1-30 wt % based on the total weight of the hard tissue regeneration material. Most preferably, the content of the ZnO particles is 2-20 wt % based on the total weight of the hard tissue regeneration material. If the content of the ZnO particles is less than the aforementioned range, a desirable anti-bacterial property of the hard tissue regeneration material may not be obtained. If the content of the ZnO particles is higher than the aforementioned range, the polycarbnoxylate cement may be hard to mix, and the adhesion between the hard tissue regeneration material and the tissues may be decreased.

Other objects, advantages, and novel features of the invention will become more apparent from the following detailed description when taken in conjunction with the accompanying drawings.

### BRIEF DESCRIPTION OF THE DRAWINGS

FIG. 1 is a result showing the biocompatibility of hard tissue regeneration materials according to Embodiments 1-4 and Comparative embodiment 1 of the present invention;

FIG. 2 is a result showing the compressive strength of hard tissue regeneration materials according to Embodiments 1-4 and Comparative embodiment 1 of the present invention;

FIG. 3 is a result showing the diametral tensile strength of hard tissue regeneration materials according to Embodiments 5-11 and Comparative embodiment 2 of the present invention;

FIG. 4 is a result showing the compressive strength of hard tissue regeneration materials according to Embodiments 5-11 and Comparative embodiment 2 of the present invention;

FIG. 5 is a result showing the diametral tensile strength of hard tissue regeneration materials according to Embodiments 12-14 and Comparative embodiment 3 of the present invention;

FIG. 6 is a result showing the compressive strength of hard tissue regeneration materials according to Embodiments 12-14 and Comparative embodiment 3 of the present invention;

FIG. 7 is a result showing the anti-bacteria property of hard tissue regeneration materials of the present invention; and

FIG. 8 is a result showing the biocompatibility of hard tissue regeneration materials according to Embodiments 12-14 and Comparative embodiment 3 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Preparation of Type I Collagen Powders

Herein, fibers of flexor digitorum profundus tendon from cattle were extracted with acetic acid solution to prepare collagen. After dialysis, the extracted collagen was precipitated with 3-4 wt % NaCl solution, and the precipitates were dried at 4° C. to obtain type I collagen powders. The dried collagen powders were stored in liquid nitrogen. In addition, SDS-PAGE and Western blot were performed to identify the purity of the obtained collagen.

### Preparation of ZnO Nanorods

Herein, a chemical bath deposition method was used to prepare ZnO nanorods.

Under stirring, 0.1 M of Zn(NO₃)₂ solution (Aldrich) was added into 0.1 M hexamethyleneteramine solution, and then white precipitates were formed by ZnO nucleation. Next, the mixture was placed in an oven at 95° C. for 8 hr to form ZnO crystals. After the crystal formed, the un-reacted starting materials were removed by a centrifuge at 3500 rpm for 10 min. The obtained ZnO nanorods were washed with distilled water and ethanol, and then dried.

The SEM photo of the ZnO nanorods (data not shown) indicates that the synthesized ZnO nanorods are hexagon-shaped nanorods, and the cross-sectional diameters thereof are 200-500 nm.

### Preparation of ZnO Hollow Fibers

Herein, a template-based method was used to prepare ZnO hollow fibers.

Cotton fibers (Consumed, 5 cm×5 cm) were used as templates, and dipped into 3.5 wt % of zinc acetate solution (JTBaker). The products were placed at 50° C. for 2 hr to remove water. Then, the cotton fibers coated with zinc acetate were placed in an oven at 600° C., sintered under normal atmosphere for 2 hr, and slowly cooled to room temperature. During sintering process, the cotton fibers were degraded into CO₂, CO, water or other easily volatile hydrocarbon. Hence, nano-ZnO hollow fibers can be obtained.

The SEM photo of the obtained ZnO hollow fibers (data not shown) indicates that the ZnO hollow fibers has tube-like structure, and the cross-sectional diameters of the ZnO hollow fibers is about 1-2 μm. In addition, the SEM photo thereof further indicates that each ZnO hollow fiber is composed of plural ZnO nanoparticles with diameters of 50-100 nm.

### Preparation of ZnO Nanoparticles (ZnO NPs)

0.1 M of zinc nitrate was mixed with 0.2 M NaOH, and the mixture was stirred at room temperature for 2 hr. The white precipitates were washed with water and the separated with a centrifuge at 3000 rpm for 5 min. After the supernatant was removed, 100 ml of H₂O₂ solution was mixed with the precipitates. The mixture was kept at 75° C. for 1 hr to obtain a sol-gel. After the sol-gel was dried and sintered at 350° C. for 6 hr, ZnO nanoballs (i.e. ZnO nanoparticles) were obtained.

Alternatively, 13.719 g of zinc acetate was dissolved in 250 ml of methanol, and the mixture was refluxed at 60° C. for 3 hr. Then, the mixture was dried under low pressure, and the dried gel was sintered at 800° C. for 3 hr. After the aforementioned process, ZnO nanoparticles were obtained.

### Embodiment 1

HY-Bond polycarboxylate cement powders (Shofu, Kyoto, Japan) (curing material) were mixed with ZnO nanorods, wherein the additive amount of the ZnO nanorods was 1 wt % based on the total amount of the powders comprising the HY-Bond polycarboxylate cement powders and the ZnO nanorods. Next, a mixing liquid, which was a liquid adhesive for the HY-Bond polycarboxylate cement powders, was mixed with the powders to form a hard tissue regeneration material.

After the aforementioned process, a hard tissue regeneration material was obtained, which comprises: HY-Bond polycarboxylate cement powders and ZnO nanorods, wherein the content of the ZnO nanorods is 1 wt % based on the total amount of the HY-Bond polycarboxylate cement powders and the ZnO nanorods.

### Embodiment 2

The method and the steps for manufacturing the hard tissue regeneration material of the present embodiment are the same as those illustrated in Embodiment 1, except that the additive amount of the ZnO nanorods was 5 wt % based on the total amount of the powders.

### Embodiment 3

The method and the steps for manufacturing the hard tissue regeneration material of the present embodiment are the same as those illustrated in Embodiment 1, except that the additive amount of the ZnO nanorods was 10 wt % based on the total amount of the powders.

### Embodiment 4

The method and the steps for manufacturing the hard tissue regeneration material of the present embodiment are the same as those illustrated in Embodiment 1, except that the additive amount of the ZnO nanorods was 15 wt % based on the total amount of the powders.

### Comparative Embodiment 1

The method and the steps for manufacturing the hard tissue regeneration material of the present embodiment are the same as those illustrated in Embodiment 1, except that only HY-Bond polycarboxylate cement powders were used to prepare the hard tissue material of the present comparative embodiment, and the ZnO nanorods were not added therein.

### Embodiments 5-11

The method and the steps for manufacturing the hard tissue regeneration material of the present embodiment are the same as those illustrated in Embodiment 1, except that the curing material used herein was glass ionomer cement (GIC) powders and the mixing liquid was a liquid adhesive for glass ionomer cement. In each embodiment of Embodiments 5-11, the additive amounts of ZnO nanoparticles were respectively 0.5, 1, 2, 5, 10, 15 and 20 wt % based on the total amount of the powders.

Hence, the hard tissue regeneration materials of Embodiments 5-11 respectively comprise: glass ionomer cement and ZnO nanoparticles; wherein the contents of the ZnO nanoparticles are respectively 0.5, 1, 2, 5, 10, 15 and 20 wt % based on the total amount of the glass ionomer cement powders and the ZnO nanoparticles.

### Comparative Embodiment 2

The method and the steps for manufacturing the hard tissue regeneration material of the present embodiment are the same as those illustrated in Embodiments 5-10, except that only glass ionomer cement powders were used to prepare the hard tissue material of the present comparative embodiment, and the ZnO nanoparticles were not added therein.

### Embodiments 12-14

The method and the steps for manufacturing the hard tissue regeneration material of the present embodiment are the same as those illustrated in Embodiments 5-11, except that the curing material used herein was a mixing material comprising glass ionomer cement (GIC) powders and type I collagen powders, and the additive amount of the ZnO nanoparticles was 2 wt % based on the total amount of the glass ionomer cement powders and the ZnO nanoparticles. In addition, the content of type I collagen powders was respectively 0.01, 0.1, and 1 wt % based on the glass ionomer powders in each embodiments.

Hence, the hard tissue regeneration materials of Embodiments 12-14 respectively comprise: type I collagen, glass ionomer cement and ZnO nanoparticles, wherein the contents of the type I collagen are respectively 0.01, 0.1 and 1 wt % based on the total amount of the glass ionomer cement powders, and the contents of the ZnO nanoparticles in each embodiment are 2 wt % based on the total amount of the hard tissue regeneration material powders.

### Comparative Embodiment 3

The method and the steps for manufacturing the hard tissue regeneration material of the present embodiment are the same as those illustrated in Embodiments 12-14, except that only glass ionomer cement powders were used to prepare the hard tissue material of the present comparative embodiment, and the ZnO nanoparticles and type I collagen powders were not added therein.

### MTT Assay

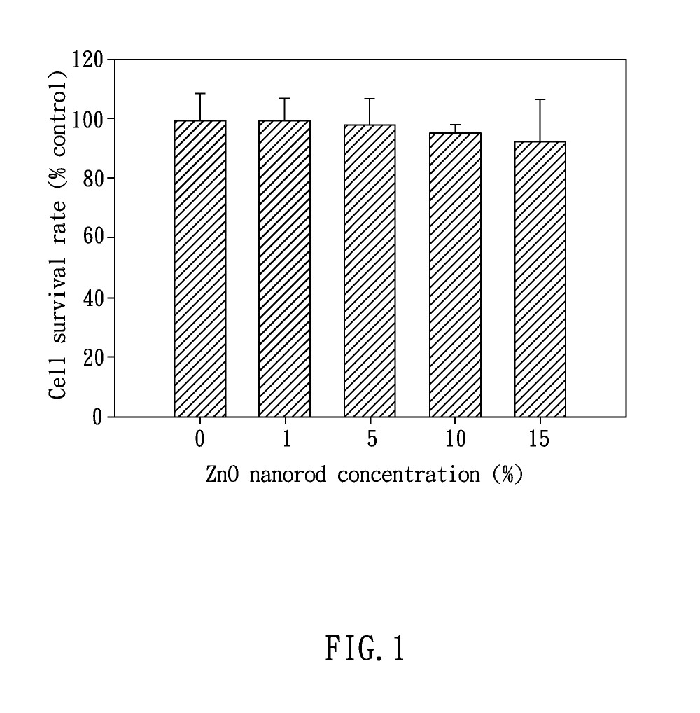

Herein, mouse fibroblast cells NIH 3T3 were used to perform the MTT assay. A required amount of prepared ZnO nanorods (as shown in Embodiments 1-4) was mixed with the HY-Bond polycarboxylate cement powders, and then a mixing liquid was mixed with the powders. The obtained slurry was pressed into specimens with 5 mm diameter and 1.5 mm thickness by using a stainless mold (i.e. hard tissue regeneration materials of Embodiments 1-4 and Comparative embodiment 1). Both sides of the specimens were respectively exposed under UV for 2 hr to perform sterilization. Then, the specimens were dipped into culture medium. NIH 3T3 cells were cultured in the culture medium extracted from the specimens and then MTT assay was performed to detect the cell survival rate. The result is shown in FIG. 1.

In FIG. 1, the cell survival rate tested by the hard tissue regeneration material of Comparative embodiment 1 is considered as 100%. Even though the additive of the ZnO nanorods is 15 wt % based on the total weight of the hard tissue regeneration material, 95% of cell survival rate still can be achieved. This result indicates that the hard tissue regeneration material containing ZnO nanorods does not damage tissues around filling areas.

### Compressive Strength Test

According to the aforementioned process, the hard tissue regeneration materials of Embodiments 1-4 and Comparative embodiment 1 were pressed into specimens with 6 mm diameter and 12 mm thickness by using a stainless mold. According to the ADA 66 standard, the obtained specimens were tested by Universal Testing Machine (Shimadzu AGS-IS, Tokyo, Japan) with a 1 mm/min testing rate.

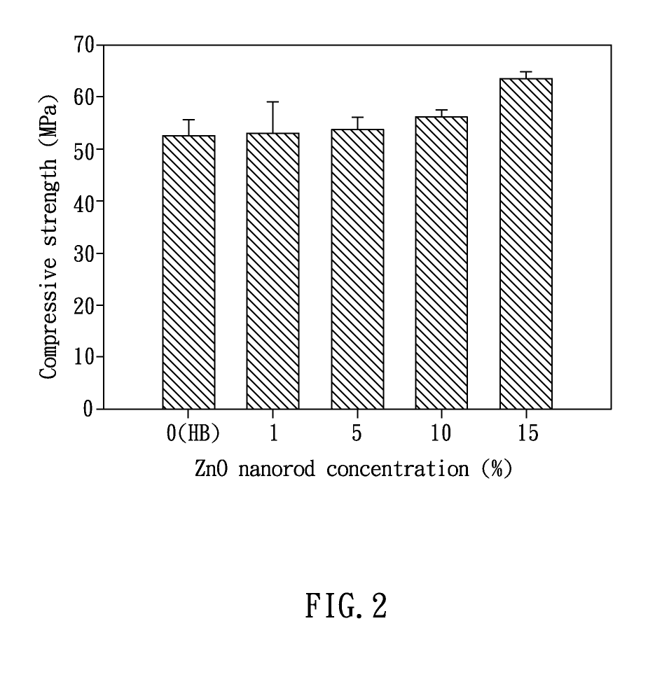

The results of the compressive strength test to the hard tissue regeneration material of Embodiments 1-4 and Comparative embodiment 1 are shown in FIG. 2.

As shown in FIG. 2, the compressive strength of the hard tissue regeneration material is improved as the additive amount of the ZnO nanorods increased. This result indicates that the ZnO nanorods can not only provide anti-bacteria effect but also improve the mechanical strength of the hard tissue regeneration material containing the same.

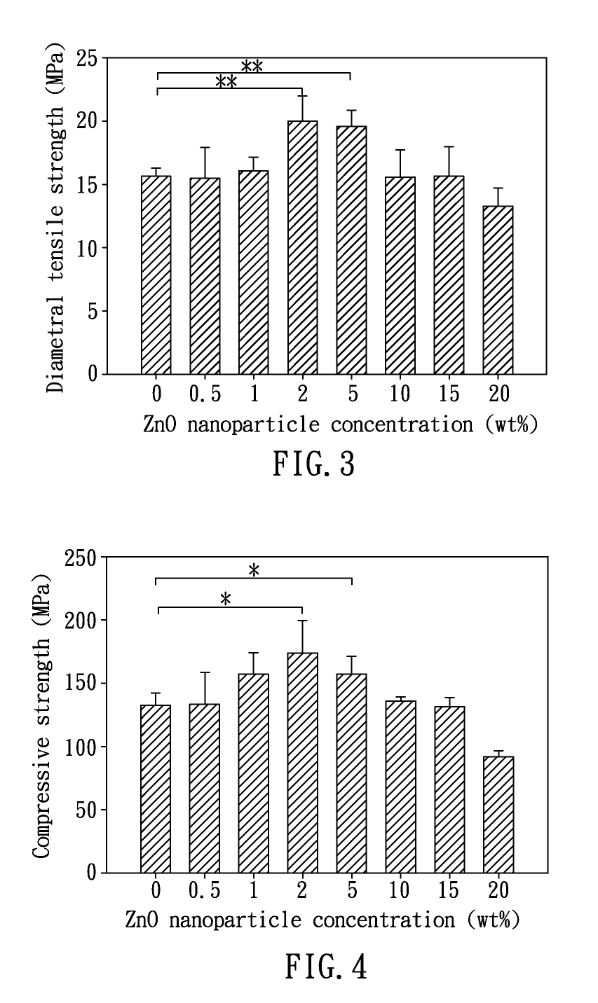

In addition, the diametral tensile strength of the hard tissue regeneration materials of Embodiments 5-11 and Comparative embodiment 2 was also examined, and the results thereof are shown in FIG. 3 and FIG. 4. These results indicate that the diametral tensile strength and the compressive strength of the hard tissue regeneration materials are improved as the additive amount of the ZnO nanoparticles increased. Especially, a significant improvement on the diametral tensile strength and the compressive strength thereof (i.e. mechanical strength thereof) can be observed when the additive amount of the ZnO nanoparticles is in a range from 2 wt % to 5 wt %.

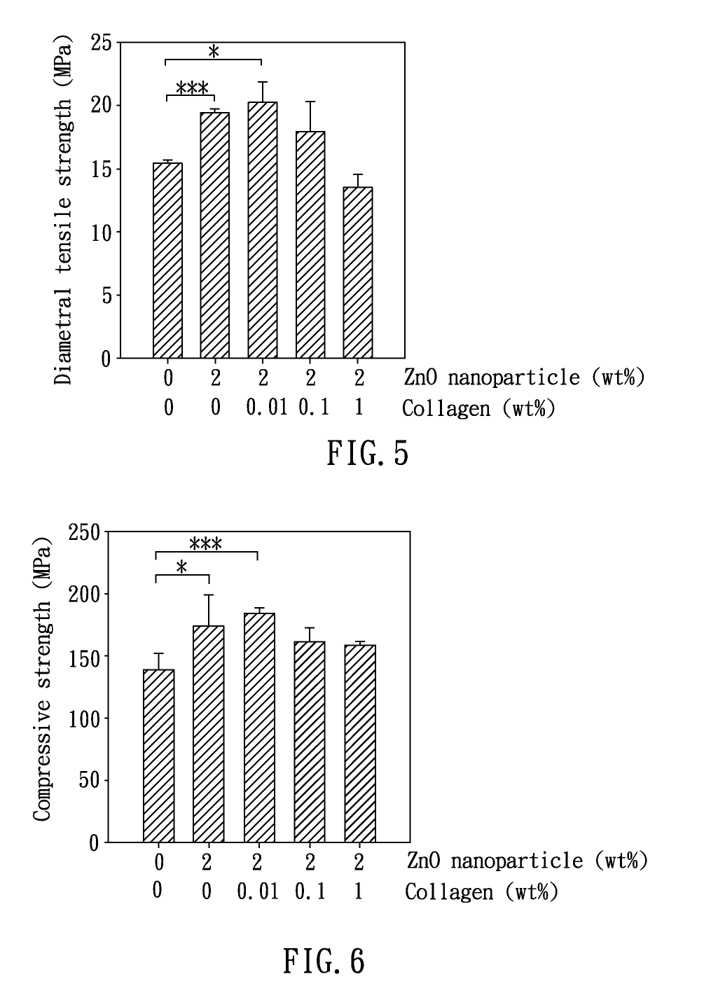

Furthermore, the diametral tensile strength and the compressive strength of the hard tissue regeneration materials of Embodiments 12-14 and Comparative embodiment 3 were also examined, and the results thereof are shown in FIG. 5 and FIG. 6. These results indicate that the diametral tensile strength and the compressive strength of the hard tissue regeneration materials are improved as the additive amount of the collagen increased. Especially, a significant improvement on the diametral tensile strength and the compressive strength thereof (i.e. mechanical strength thereof) can be observed when the additive amount of the collagen is 0.01 wt % and that of the ZnO nanoparticles is 2 wt %.

The aforementioned results indicate that the mechanical strength of the hard tissue regeneration material can be improved by using not only ZnO nanorods or ZnO nanoparticles but also suitable amount of collagen.

### Anti-Bacteria Test

The anti-bacteria property of the hard tissue regeneration materials of Embodiments 1-4 and Comparative embodiment 1 was examined by using Streptococcus mutants (S. mutants) B215.

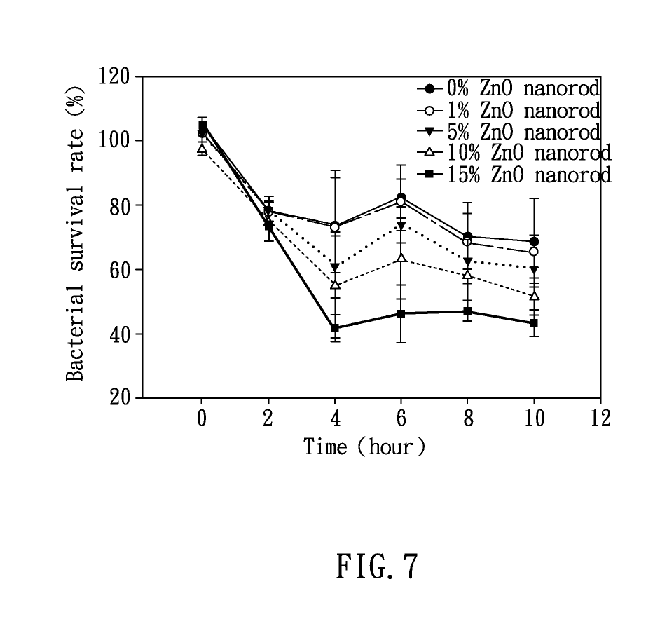

Briefly, the uncured hard tissue regeneration materials of Embodiments 1-4 and Comparative embodiment 1 were filled into Teflon molds with 5 mm diameter and 1.5 mm thickness to form solidified specimens. Both sides of the specimens were respectively exposed under UV for 2 hr to perform sterilization. The sterilized specimens were transferred into 96-wells plate, and then 500 μl of Streptococcus mutants suspension was added therein. The initial OD_{600 nm} of the Streptococcus mutants solution is 0.03. The bacteria survival rate of the Streptococcus mutants cultured with the specimens prepared by the hard tissue regeneration materials of Embodiments 1-4 and Comparative embodiment 1 were examined every two hours. The results are shown in FIG. 7.

As shown in FIG. 7, the bacterial survival rate was decreased as the additive amount of ZnO nanorods increased. When the additive amount of the ZnO nanorods was more than 5 wt % (Embodiment 2), the bacterial survival rate was significantly decreased. Especially, the bacterial survival rate was decreased to less than 50% when the additive amount of the ZnO nanorods was more than 15 wt %.

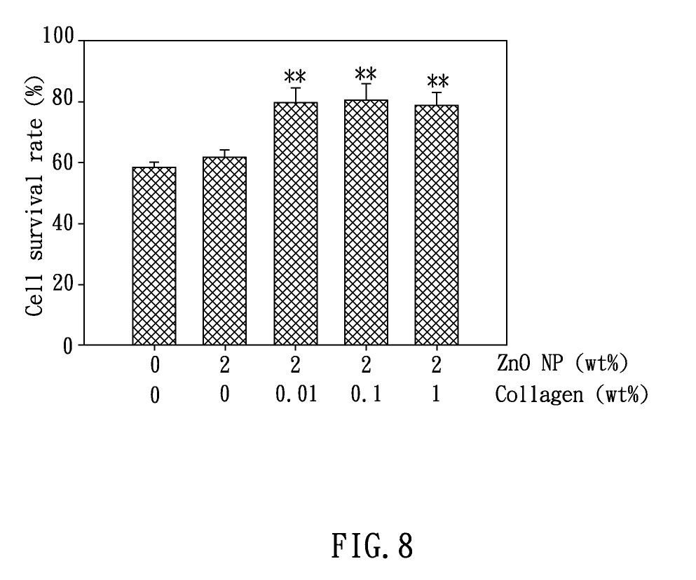

In addition, the biocompatibility of the hard tissue regeneration materials of Embodiments 12-14 and Comparative embodiment 3 was also examined according to the process illustrated above. The results are shown in FIG. 8.

In conclusion, a suitable amount of ZnO particles can improve the mechanical strength of the hard tissue regeneration material of the present invention without decreasing the biocompatibility thereof. In addition, the hard tissue regeneration material of the present invention can also have anti-bacteria property by adding a suitable amount of ZnO particles. Therefore, since the hard tissue regeneration material of the present invention have anti-bacteria property, so it can further be applied to mechanical field such as dental treatment.

Although the present invention has been explained in relation to its preferred embodiment, it is to be understood that many other possible modifications and variations can be made without departing from the spirit and scope of the invention as hereinafter claimed.

What is claimed is:

1. A method for manufacturing a hard tissue regeneration material, comprising the following steps:
(A) providing a curing material and ZnO particles wherein the curing material is at least one selected from the group consisting of polycarboxylate cement, glass ionomer cement, and collagen, the ZnO particles are at least one selected from the group consisting of crystallized ZnO nanoparticles, crystallized ZnO nanorods, and nano-ZnO hollow fibers, diameters of the crystallized ZnO nanoparticles are 25 nm-200 nm, cross-sectional diameters of the crystallized ZnO nanorods are 50 nm-1000 nm, the nano-ZnO hollow fibers have tube-like structure, and cross-sectional diameters of the nano-ZnO hollow fibers is 500 nm-3 μm; and
(B) mixing the curing material and the ZnO particles to form a hard tissue regeneration material.

2. The method as claimed in claim 1, wherein each nano-ZnO hollow fibers is composed of plural ZnO nanoparticles.

3. The method as claimed in claim 2, wherein diameters of the ZnO nanoparticles are 20 nm-200 nm.

4. The method as claimed in claim 1, wherein the crystallized ZnO nanoparticles are single-crystallized ZnO nanoparticles, poly-crystallized ZnO nanoparticles, or a combination thereof.

5. The method as claimed in claim 1, wherein the polycarboxylate cement is HY-Bond polycarboxylate cement.

6. The method as claimed in claim 1, wherein a content of the ZnO particles is 0.01-40 wt % based on a total weight of the hard tissue regeneration material.

7. A hard tissue regeneration material, comprising:
a curing material, which is at least one selected from the group consisting of polycarboxylate cement, glass ionomer cement, and collagen; and
ZnO particles, which are at least one selected from the group consisting of crystallized ZnO nanoparticles, crystallized ZnO nanorods, and nano-ZnO hollow fibers, wherein diameters of the crystallized ZnO nanoparticles are 25 nm-200 nm, cross-sectional diameters of the crystallized ZnO nanorods are 50 nm-1000 nm, the nano-ZnO hollow fibers have tube-like structure, and cross-sectional diameters of the nano-ZnO hollow fibers is 500 nm-3 μm.

8. The hard tissue regeneration material as claimed in claim 7, wherein the polycarboxylate cement is HY-Bond polycarboxylate cement.

9. The hard tissue regeneration material as claimed in claim 7, wherein a content of the ZnO particles is 0.01-40 wt % based on a total weight of the hard tissue regeneration material.

10. The hard tissue regeneration material as claimed in claim 7, wherein each nano-ZnO hollow fiber is composed of plural ZnO nanoparticles.

11. The hard tissue regeneration material as claimed in claim 10, diameters of the ZnO nanoparticles are 20 nm-200 nm.

12. The hard tissue regeneration material as claimed in claim 7, wherein the crystallized ZnO nanoparticles are single-crystallized ZnO nanoparticles, poly-crystallized ZnO nanoparticles, or a combination thereof.
